(19) United States
(12) Patent Application Publication
(10) Pub. No.: US 2021/0327187 A1
(43) Pub. Date: Oct. 21, 2021

(54) MEDICAL SCREENING ENTRY

(71) Applicant: Alclear, LLC, New York, NY (US)

(72) Inventor: Rob Wisniewski, New York, NY (US)

(21) Appl. No.: 17/232,555

(22) Filed: Apr. 16, 2021

Related U.S. Application Data
(60) Provisional application No. 63/011,676, filed on Apr. 17, 2020.
(60) Provisional application No. 63/053,021, filed on Jul. 17, 2020.

(51) Int. Cl. G07C 9/25; G16H 10/20; G16H 10/60; G06K 19/06; G07C 9/27; H04L 29/06

(52) U.S. Cl. CPC G07C 9/257; G16H 10/20; G16H 10/60; G16H 40/67; G07C 9/27; H04L 63/08; G06K 19/06037

(57) ABSTRACT

A medical screening system includes one or more request devices, screening servers, and measurement and/or other testing devices. These devices communicate to receive one or more requests for screening, perform the screening, obtain measurements and/or test results for the screening, and allow and/or deny access based on the screening. In some examples, the testing devices may be home test kits. In various examples, the measurements and/or test results may be verified and/or verified as corresponding to a particular identity. In a number of examples, different confidence levels may be determined using different measurements and/or test results and different access may be allowed and/or denied based on the different determined confidence levels.

### CROSS-REFERENCE TO RELATED APPLICATION(S)

This application is a nonprovisional patent application of and claims the benefit of U.S. Provisional Patent Application No. 63/011,676, filed Apr. 17, 2020 and titled “Medical Screening Entry,” U.S. Provisional Patent Application No. 63/053,021, filed Jul. 17, 2020 and titled “Medical Screening Entry,” the disclosures of which are hereby incorporated herein by reference in their entireties.

### FIELD

The described embodiments relate generally to screening. More particularly, the present embodiments relate to medical screening entry.

### BACKGROUND

Access is controlled to a variety of different places and/or situations. For example, ticket takers may control access to event venues, such as sporting arenas. By way of another example, airport gate personnel control entrance onto flights. In yet another example, medical service personnel control access to areas where medical services are provided.

Access may be controlled according to a medical status of people. For example, lifeguards prohibit people with communicable illnesses from using public pools. If a lifeguard observes that a person has the symptoms of a communicable illness, the lifeguard may deny the person access to the public pool.

### SUMMARY

The present disclosure relates to medical screening systems. One or more request devices, screening servers, and measurement and/or other testing devices communicate to receive one or more requests for screening, perform the screening, obtain measurements and/or test results for the screening, and allow and/or deny access based on the screening. In some examples, the testing devices may be home test kits. In various examples, the measurements and/or test results may be verified and/or verified as corresponding to a particular identity. In a number of examples, different confidence levels may be determined using different measurements and/or test results and different access may be allowed and/or denied based on the different determined confidence levels.

In various embodiments, a system for medical screening entry includes an application operable to execute on a request device, a screening server, and a measurement device. The application receives a request for screening from a person. The application communicates with the screening server to perform a screening interaction. The application receives a measurement identifier from the screening server. The application provides the measurement identifier to the measurement device. The measurement device obtains a measurement from the person The measurement device registers the measurement with a screening record for the person. The application receives a screening access identifier from the screening server. The application provides the screening access identifier to an access control station.

In some examples, the measurement identifier is a Quick Read code. In various examples, the screening access identifier is a Quick Read code.

In some embodiments, a system for medical screening entry includes an application operable to execute on a request device, a screening server, an identity system, and a measurement device. The application receives a request for screening from a person. The application communicates with the identity system to obtain an authentication for the person. The application communicates with the screening server to perform a screening interaction wherein the application provides the authentication to the screening server. The application receives a measurement identifier from the screening server. The application provides the measurement identifier to the measurement device. The measurement device obtains a measurement from the person. The measurement device registers the measurement with a screening record for the person. The application receives a screening access identifier from the screening server. The application provides the screening access identifier to an access control station.

In various examples, personal health information is omitted from the measurement identifier. In some examples, personal identification information is omitted from the measurement identifier. In a number of examples, the measurement device obtains the measurement using an integrated sensor. In various examples, the measurement device obtains the measurement using a connected sensor. In some examples, the measurement device obtains the measurement from a manual entry obtained from an operator via an interface.

In a number of embodiments, a system for medical screening entry includes an application operable to execute on a request device, a screening server, an identity system, and a measurement device. The application receives a request for screening from a person. The application communicates with the identity system to obtain an authentication for the person and add information for the person to a local gallery. The application communicates with the screening server to perform a screening interaction wherein the application provides the authentication to the screening server. The measurement device uses the local gallery to obtain an identification for the person. The measurement device obtains a measurement from the person. The measurement device registers the measurement with a screening record for the person. The application receives a screening access identifier from the screening server. The application provides the screening access identifier to an access control station.

In some examples, the local gallery includes a subset of biometric data stored by the identity system. In various examples, the local gallery is stored by the identity system. In a number of examples, the local gallery is stored local to the measurement device. In some examples, the identity system provides the authentication in response to receiving a digital representation of a biometric for the person from the application.

In various embodiments, a system for medical screening entry includes an application operable to execute on a request device, a screening server, an identity system, and a measurement device. The application receives a request for screening from a person. The application communicates with the identity system to obtain an authentication for the person and add information for the person to a local gallery. The application communicates with the screening server to perform a screening interaction wherein the application provides the authentication to the screening server. The measurement device uses the local gallery to obtain an identification for the person. The measurement device obtains a measurement from the person. The measurement device registers the measurement with a screening record for the person. The measurement device communicates with an access control station to control access.

In some examples, the measurement device is integrated into the access control station. In a number of examples, the measurement device communicates the identification to the screening server to determine the screening record with which to register the measurement. In various examples, the measurement device communicates the identification to the screening server to determine the measurement to obtain.

In a number of examples, the measurement device uses the local gallery upon obtaining a digital representation of a biometric for the person. In some implementations of such examples, the digital representation of the biometric is at least one of a retinal image, an iris image, or a facial image.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example system for medical screening entry.

FIG. 2 depicts a second example system for medical screening entry.

FIG. 3 depicts a third example system for medical screening entry.

FIG. 4 depicts a fourth example system for medical screening entry.

FIG. 5 depicts a fifth example system for medical screening entry.

FIG. 6 is a flow chart illustrating a first example method for medical screening entry. This method may be performed by one or more of the systems of FIGS. 1-5 and 10.

FIG. 7 is a flow chart illustrating a second example method for medical screening entry. This method may be performed by one or more of the systems of FIGS. 1-5 and 10.

FIG. 8 is a flow chart illustrating a third example method for medical screening entry. This method may be performed by one or more of the systems of FIGS. 1-5 and 10.

FIG. 9 is a flow chart illustrating a fourth example method for medical screening entry. This method may be performed by one or more of the systems of FIGS. 1-5 and 10.

FIG. 10 illustrates example relationships among example components that may be used to implement one or more of the systems of FIGS. 1-5 and 10.

FIG. 11 is a flow chart illustrating a fifth example method for medical screening entry. This method may be performed by one or more of the systems of FIGS. 1-5 and 10.

FIG. 12 is a flow chart illustrating a sixth example method for medical screening entry. This method may be performed by one or more of the systems of FIGS. 1-5 and 10.

FIG. 13 is a flow chart illustrating a seventh example method for medical screening entry. This method may be performed by one or more of the systems of FIGS. 1-5 and 10.

FIG. 14 is a flow chart illustrating an eighth example method for medical screening entry. This method may be performed by one or more of the systems of FIGS. 1-5 and 10.

FIG. 15 is a flow chart illustrating a ninth example method for medical screening entry. This method may be performed by one or more of the systems of FIGS. 1-5 and 10.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, apparatuses, methods, and computer program products that embody various elements of the present disclosure. However, it should be understood that the described disclosure may be practiced in a variety of forms in addition to those described herein.

The present disclosure relates to medical screening systems. One or more request devices, screening servers, and measurement and/or other testing devices communicate to receive one or more requests for screening, perform the screening, obtain measurements and/or test results for the screening, and allow and/or deny access based on the screening. In some examples, the testing devices may be home test kits. In various examples, the measurements and/or test results may be verified and/or verified as corresponding to a particular identity. In a number of examples, different confidence levels may be determined using different measurements and/or test results and different access may be allowed and/or denied based on the different determined confidence levels.

In this way, the systems are able to protect and restrict access to private information, facilitate efficient communication between components, and ensure that information exchanged is accurate and verified. This may allow performance of functions that were previously not performable and enables more efficiently while expending less work, eliminating unnecessary hardware and/or other components, and more efficiently using hardware, software, network, and/or other resources. This may improve the operation of systems involved by reducing unnecessary components, increasing the speed at which the systems perform operations, and/or reducing consumption of hardware, software, network, and/or other resources.

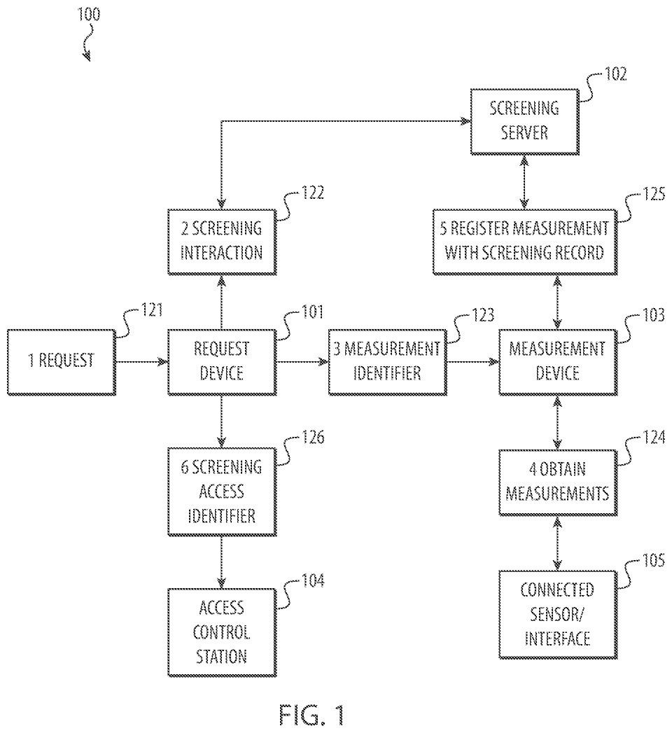

These and other embodiments are discussed below with reference to FIGS. 1-15. However, those skilled in the art will readily appreciate that the detailed description given herein with respect to these Figures is for explanatory purposes only and should not be construed as limiting.

FIG. 1 depicts a first example system 100 for medical screening entry. A request device 101 may receive a request 121 for screening from a user. The request 121 may authenticate the user, such as by including a login and/or password, an identifier, and so on. The request device 101 may engage in a screening interaction 122 with a screening server 102. The screening server 102 may create and/or update a screening record as part of the screening interaction 122. For example, the request device 101 and the screening server 102 may communicate in order to prompt the user to provide answers to one or more automated questions, to perform a telemedicine screening with the user, and so on. The screening may involve obtaining a measurement from the user and/or performing any kind of test or tests on a user to obtain one or more test results, such as a temperature, an antibody test, a white blood cell count, a thermal scan, a blood test, a saliva test, a breath test, and so on. The screening server 102 may provide a measurement identifier 123 to the request device 101, which then may provide the measurement identifier 123 to a measurement device. In some examples, the measurement identifier 123 may be an optical code (such as a Quick Read code or “QR code” and/or other bar code). The measurement identifier 123 may include information usable to reconcile a screening and/or screening request with a measurement. The measurement identifier 123 may omit personal health information for the user, personal identifying information for the user, and so on. The measurement device 103 may obtain the measurement 124 (such as a temperature, an antibody test, a white blood cell count, a thermal scan, a blood test, a saliva test, a breath test, and so on) from the user using a connected sensor (such as a thermometer, an antibody tester, a blood sensor, a thermal sensor, a thermal camera, a thermal imaging sensor, a saliva sensor, a breath sensor, and so on) and/or an interface 105 usable by an operator to manually enter the measurement 124 based on results obtained by the operator. The measurement device 103 may use the information in the measurement identifier 123 to ascertain information to associate with the measurement 124, determine the measurement 124 to obtain, and so on. The measurement device 103 may communicate with the screening server 102 accordingly to register the measurement with the screening record 125. The screening server 102 may then provide a screening access identifier 126 (whether to allow access, deny access, conditionally allow access, allow a particular access out of a set of possible accesses, and so on), which the request device 101 may provide at and/or to an access control station 104. In some examples, the screening access identifier 126 may be an optical code (such as QR code and/or other bar code). The screening access identifier 126 may include information usable to determine the access to allow and/or deny. The measurement identifier 123 may omit personal health information for the user, personal identifying information for the user, and so on. The access control station 104 may allow and/or deny access accordingly.

For example, a user may have a screening system app on his phone and/or other mobile computing device. The user may authenticate himself to the app and request screening. The user's phone may communicate with a screening system backend to answer automated questions and/or perform a telemedicine screening to create and/or update a screening record for the user. The screening may require a temperature reading. The screening system backend may provide the user's phone a measurement QR code (and/or information that the user's phone may use to generate such a measurement QR code) that the user may scan via a screener's tablet or other computing device. This may inform the screener's tablet to obtain a temperature reading from the user. The screener's tablet may use a connected thermometer (and/or other thermal sensor, a thermal camera, a thermal imaging sensor, and so on) (and/or the screener may use a separate thermometer and/or other thermal sensor, a thermal camera, a thermal imaging sensor, and so on to obtain the temperature reading and manually enter the temperature reading into the screener's tablet via an interface) to obtain the temperature from the user and use the information from the measurement QR code to communicate with the screening system backend to register the thermometer reading with the appropriate screening record. In response to registration of the thermometer reading, the screening system backend may provide the user's phone a screening access QR code. For example, if the temperature reading indicates that the user does not have a fever, the screening access QR code may indicate to allow access. By way of another example, if the user does have a fever, the screening access QR code may indicate to deny access. In yet another example, the screening access QR code may indicate to allow unfettered access if the user does not have a fever and access to an isolated area if the user does have a fever. The user may scan the screening access QR code at an automated gate, may show the screening access QR code to gate personnel, and so on.

For example, the system 100 may be used to control access to a medical testing lab, medical waiting room, and/or other medical service provider facility. The system 100 may also be used to control access to an event venue, a restaurant, a nursing home, a secured building, an airport and/or other transportation facility, a gym and/or other fitness and/or training facility, and/or any other area. Various configurations are possible and contemplated without departing from the scope of the present disclosure.

In yet another example, the access control station 104 may be a station at a gym and/or other fitness and/or training facility. The station may include components that obtain a digital representation of a biometric for a person. The station may use the digital representation of the biometric to access health information for the person and determine whether or not the health information indicates that the person might have a communicable disease. If not, the station may allow the person access to the gym and/or other fitness and/or training facility. Otherwise, the person may be denied access. In some implementations, the station may determine a confidence level of the determination and may allow different levels of access for different determined confidence levels. By way of illustration, a person with a low confidence level may be allowed access conditional to use of protective gear (such as a mask) and temperature or other health information verification, a person with a middle confidence level may be allowed access conditional to use of protective gear without a temperature or other health information verification, and a person with a high confidence level may be allowed access without use of protective gear and/or temperature or other health information verification. Various configurations are possible and contemplated without departing from the scope of the present disclosure.

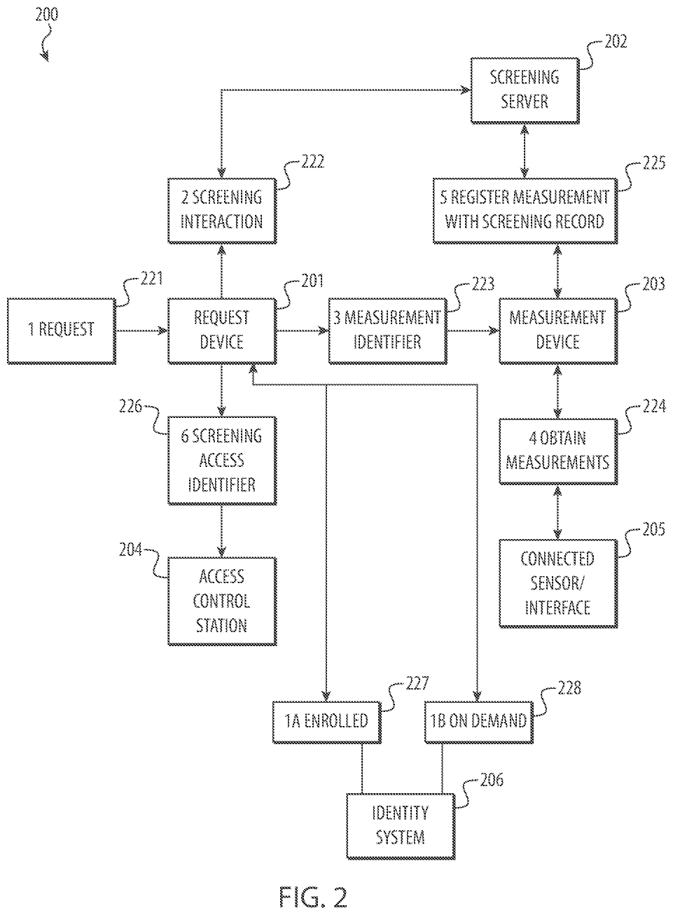

FIG. 2 depicts a second example system 200 for medical screening entry. The system 200 is similar to the system 100 of FIG. 1 in that the request device 201 communicates with a screening server 202 (such as for a screening interaction 222) based on a screening request 221, provides a measurement identifier 223 to a measurement device 203 that obtains the measurement 224 (such as via a connected sensor/interface 205) and registers the measurement 225 with the screening server 202, provides a screening access identifier 226 obtained from the screening server 202 based on the measurement 224, and enables access via an access control station 204. The system 200 differs from the system 100 of FIG. 1 at least in that the system 100 may rely on the user to self-authenticate to the screening server 102 via the request device 101 whereas in the system 200 the request device 201 is operable to communicate with an identity system 206 in order for the identity system 206 to identify and/or authenticate the user instead of the user identifying and/or authenticating himself.

In some implementations, the identity system 206 may be operable to identify and/or authenticate users who are either enrolled and/or not enrolled with the identity system 206. The identity system 206 may store identification information, such as digital representations of one or more biometrics (which may include one or more digital representations of one or more fingerprints, blood vessel scans, palm-vein scans, voiceprints, facial images, retina images, iris images, deoxyribonucleic acid sequences, heart rhythms, gaits, and so on), logins and/or passwords, and so on. The identity system 206 may associate the identification information with identity information (such as one or more names, addresses, telephone numbers, social security numbers, patient identification numbers or other identifiers, insurance data, financial data, health information (such as one or more temperatures, pupil dilation, medical diagnoses, immunocompromised conditions, medical histories, medical records, infection statuses, vaccinations, immunology data, results of antibody tests evidencing that a person has had a particular communicable illness and recovered, blood test results, saliva test results, and/or the like, though health information may be stored separately from identity information in some implementations, such as via one or more identifiers for such health information that may be stored in and/or otherwise associated with the identity information, such as in a Health Insurance Portability and Accountability Act (“HIPAA”) compliant or other data store or enclave and/or a blockchain and/or other auditable record or ledger). The request device 201 may include a biometric reader (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on) that may be used to obtain one or more digital representations of one or more biometrics from the user and provide such to the identity system 206. If the user is enrolled 227, the identity system 206 may compare the digital representation of the biometric to stored identification information to identify and/or authenticate the user and provide information based thereon to the screening server 202 via the request device 201 and/or directly. Alternatively, if the user is not enrolled, the identity system 206 may identify and/or authenticate the user on demand 228, such as by validating a scan of a driver's license or other identity document and comparing a picture on the driver's license to a facial image of the user captured using a camera or other 2D or 3D image sensor of the request device 201.

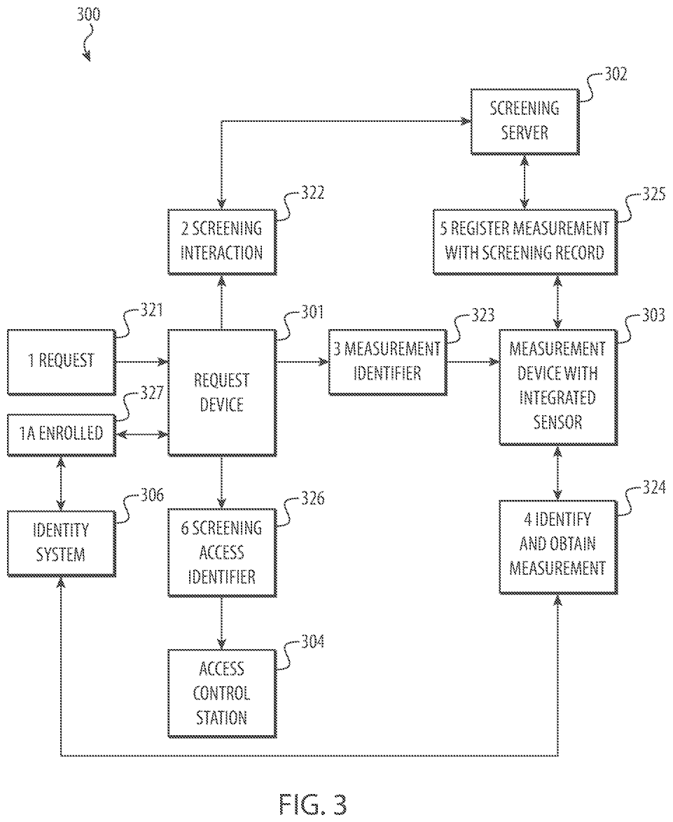

FIG. 3 depicts a third example system 300 for medical screening entry. The system 300 is similar to the system 200 of FIG. 2 in that the request device 301 communicates with a screening server 302 (such as for a screening interaction 322) based on a screening request 321, provides a measurement identifier 323 to a measurement device 303 that obtains the measurement 324 and registers the measurement 325 with the screening server 302, determines whether or not a user is enrolled 327 with an identity system 306, provides a screening access identifier 326 obtained from the screening server 302 based on the measurement 324, and enables access via an access control station 304. 327 The system 300 differs from the system 200 of FIG. 2 at least in that the measurement device 303 may communicate with the identity system 306 as part of identifying the user and obtaining the measurement 324.

For example, the measurement device 303 may obtain information from the measurement identifier 323 regarding an identification of the user. The measurement device 303 may use a camera or other 2D or 3D image sensor to obtain a facial image from the user (and/or other biometric reader or readers to obtain one or more other digital representations of one or more biometrics from the user) while (and/or previous to and/or subsequent to) the measurement is obtained. The measurement device 303 may track the user's face and/or other biometric during measurement to ensure that the measurement is obtained from the same person as the facial image and/or other biometric. The measurement device 303 may communicate with the identity system 306 to confirm that the facial image and/or other biometric corresponds to the identified person, which may be performed efficiently since the measurement device 303 may be requesting a 1:1 comparison of a particular facial image and/or other biometric to biometric information stored for a specified person. As such, the identity corresponding to the measurement may be verified by the identity system 306 as well as the identity corresponding to the request 321.

Although this is illustrated and described as the measurement device 303 communicating with the identity system 306, it is understood that this is an example. In other implementations, the screening server 302 may act as an intermediary between the measurement device 303 and the identity system 306 for such communications. Various configurations are possible and contemplated without departing from the scope of the present disclosure.

Further, the system 300 may also differ from the system 200 of FIG. 2 at least in that the measurement device 303 may include an integrated sensor and/or interface for obtaining the measurement instead of communicating with a connected sensor and/or interface like in the system 200 of FIG. 2. Various configurations are possible and contemplated without departing from the scope of the present disclosure.

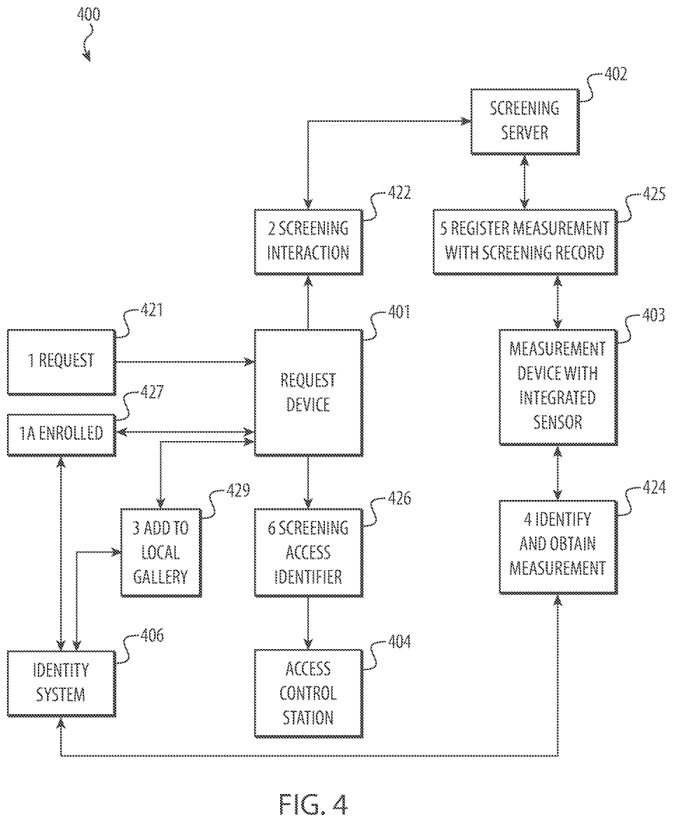

FIG. 4 depicts a fourth example system 400 for medical screening entry. The system 400 is similar to the system 300 of FIG. 3 in that the request device 401 communicates with a screening server 402 (such as for a screening interaction 422) based on a screening request 421, a measurement device 403 obtains the measurement 424 and registers the measurement 425 with the screening server 402, the request device 401 determines whether or not a user is enrolled 427 with an identity system 406, and the request device 401 provides a screening access identifier 426 obtained from the screening server 402 based on the measurement 424 and enables access via an access control station 304. The system 400 differs from the system 300 of FIG. 3 at least in that the request device 401 may not obtain a measurement identifier from the screening server 402 and provide such to the measurement device 403.

Instead, interaction between the request device 401 and the identity system 406 may prompt the identity system 406 to add information for the user to a local gallery 429 for the screening. In some examples, this may be a data store local to the measurement device 403. In other examples, this may be a data store of the identity system 406 that is segmented to dedicate a portion to the measurement device 403 and/or the screening. As such, the user may provide a digital representation of a biometric (such as a facial image and/or other digital representation of a biometric) to the measurement device (and/or previous to and/or subsequent to) while the measurement is obtained. The measurement device 403 may provide this digital representation of the biometric to the identity system 406 to identify and/or authenticate the user and/or obtain information regarding the user and/or the screening record to which to register the measurement.

As the local gallery may include users who have requested screening via the screening server 402 and are to obtain measurements via the measurement device 403, the local gallery may include only a subset of the information stored by the identity system 406 as a whole. This may allow comparison of the digital representation of the biometric to a smaller set of stored biometric information for the purposes of identification and/or authentication than that stored by the identity system 406 as a whole. This may increase accuracy of identifications and/or authentications, improve the speed of identifications and/or authentications, consume less hardware and/or software resources for identifications and/or authentications, improve system 400 efficiency, and so on.

However, it is understood that the system 400 is an example. In other implementations, an app and/or application executing on a user device (such as a smart phone, other mobile device, and so on) may obtain one or more digital representations of one or more biometrics from a person, communicate with the identity system 406 and/or one or more other devices to determine an identity of the person, prompt the person to answer one or more health-related questions (which may be selected according to a particular set of requirements associated with the access control station 404), and (dependent upon the answer(s) to the one or more health-related questions) display and/or provide one or more outputs (such as one or more QR codes) that the person may use the user device to provide to the access control station 404 and/or one or more associated devices. The access control station 404 may then allow and/or deny access based upon the provide done or more outputs and/or the associated answer(s) to the one or more health-related questions, the particular set of requirements, and/or other information stored in association with the identity. Various configurations are possible and contemplated without departing from the scope of the present disclosure.

In another example, the access control station 404 may obtain one or more digital representations of one or more biometrics from a person, communicate with the identity system 406 and/or one or more other devices to determine an identity of the person, allow and/or deny access based on one or more answers (which may be stored and/or otherwise associated with the identity information) to one or more health-related questions (which may be selected according to a particular set of requirements associated with the access control station 404), the particular set of requirements, and/or other information stored in association with the identity, and so on. Various configurations are possible and contemplated without departing from the scope of the present disclosure.

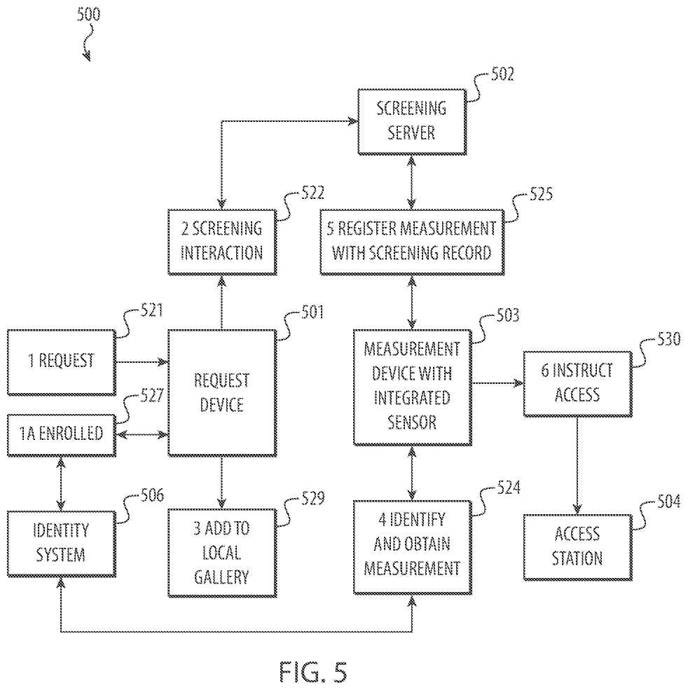

FIG. 5 depicts a fifth example system 500 for medical screening entry. The system 500 is similar to the system 400 of FIG. 4 in that the request device 501 communicates with a screening server 502 (such as for a screening interaction 522) based on a screening request 521, the request device determines whether or not a user is enrolled 527 with an identity system 506, the request device 401 and the identity system 406 may coordinate to add information for the user to a local gallery 529 for a screening, and a measurement device 503 obtains the measurement 524 and registers the measurement 525 with the screening server 502. The system 500 differs from the system 400 of FIG. 4 at least in that the request device 501 may not provide a screening access identifier obtained from the screening server 502 based on the measurement 524.

Instead, the measurement device 503 may be integrated with and/or communicate directly with the access station 504. Specifically, the measurement device 503 may instruct access 530 to the access station 504. However, it is understood that this is an example. In some implementations, the screening server 502 may act as an intermediary between the measurement device 503 and the access station 504 for such communications. Various configurations are possible and contemplated without departing from the scope of the present disclosure.

Although the systems 100-500 of FIGS. 1-5 are illustrated and described as including particular devices performing particular operations, it is understood that these are examples. Other configurations and/or operations are possible and contemplated without departing from the scope of the present disclosure.

For example the systems 100-500 of FIGS. 1-5 are illustrated and described as obtaining one or more measurements as part of screening. However, it is understood that this is an example. In some implementations, the screening server 102-502 may determine not to obtain the measurement (such as based on information obtained during the screening interaction 122-522, where data that may be substituted from the measurement is obtained from identity information stored by the identity system 206-506 and/or from health information stored in association with the identity information stored by the identity system 206-506, and so on). In such examples, obtaining the measurement 124-524 may be omitted. Various configurations are possible and contemplated without departing from the scope of the present disclosure.

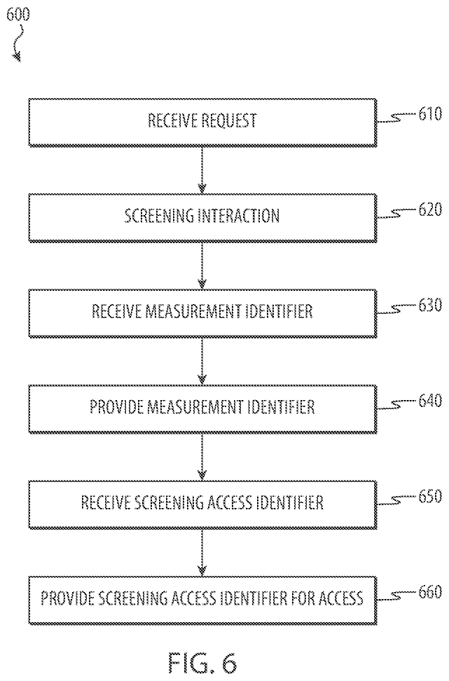

FIG. 6 is a flow chart illustrating a first example method 600 for medical screening entry. This method 600 may be performed by one or more of the systems 100-500 and 1000 of FIGS. 1-5 and 10.

At operation 610, an electronic device, such as one or more of the request devices 101-501, 1001 of FIGS. 1-5, may receive a request for screening. At operation 620, the electronic device may engage in a screening interaction between the user and a screening server. At operation 630, the electronic device may receive a measurement identifier (such as a QR code) from the screening server and/or information to use to generate the measurement identifier. At operation 640, the electronic device may provide the measurement identifier to a measurement device. At operation 650, the electronic device may receive a screening access identifier (such as a QR code) from the screening server and/or information to use to generate the screening access identifier. At operation 660, the electronic device may provide the screening access identifier for access, such as by providing the screening access identifier to an access control station.

In various examples, this example method 600 may be implemented as a group of interrelated software modules or components that perform various functions discussed herein. These software modules or components may be executed within a cloud network and/or by one or more computing devices, such as the as one or more of the request devices 101-501, 1001 of FIGS. 1-5.

Although the example method 600 is illustrated and described as including particular operations performed in a particular order, it is understood that this is an example. In various implementations, various orders of the same, similar, and/or different operations may be performed without departing from the scope of the present disclosure.

For example, in some implementations, the method 600 may include the additional operations of obtaining a digital representation of a biometric from a user and communicating with an identity system to identify and/or authenticate the user. Various configurations are possible and contemplated without departing from the scope of the present disclosure.

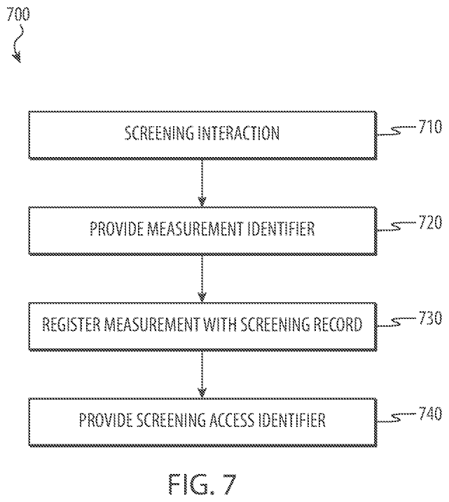

FIG. 7 is a flow chart illustrating a second example method 700 for medical screening entry. This method 700 may be performed by one or more of the systems 100-500 and 1000 of FIGS. 1-5 and 10.

At operation 710, an electronic device, such as one or more of the screening servers 102-502, 1002 of FIGS. 1-5, may engage in a screening interaction with one or more users via one or more request devices in response to one or more requests from one or more users. At operation 720, the electronic device may provide a measurement identifier (such as a QR code) and/or information to generate such to the request device based on the screening interaction. At operation 730, the electronic device may register a measurement received from one or more measurement devices with the screening record for the user. At operation 740, the electronic device may provide an access identifier (such as a QR code) and/or information to generate such to the request device.

In various examples, this example method 700 may be implemented as a group of interrelated software modules or components that perform various functions discussed herein. These software modules or components may be executed within a cloud network and/or by one or more computing devices, such as one or more of the screening servers 102-502, 1002 of FIGS. 1-5.

Although the example method 700 is illustrated and described as including particular operations performed in a particular order, it is understood that this is an example. In various implementations, various orders of the same, similar, and/or different operations may be performed without departing from the scope of the present disclosure.

For example, operation 720 is illustrated and described as providing the measurement identifier. However, it is understood that this is an example. In other implementations, a measurement device may interact with an identity system to identify a user associated with the measurement and the electronic device may use such information to register the measurement with a corresponding screening record without having provided a measurement identifier. Various configurations are possible and contemplated without departing from the scope of the present disclosure.

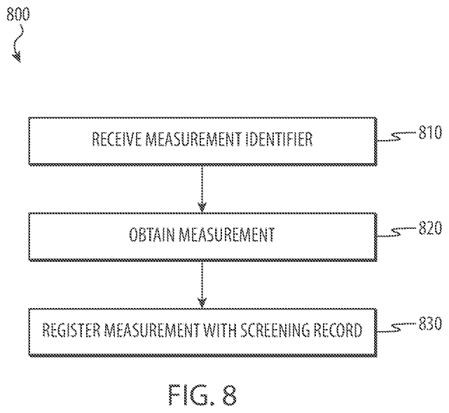

FIG. 8 is a flow chart illustrating a third example method 800 for medical screening entry. This method 800 may be performed by one or more of the systems 100-500 and 1000 of FIGS. 1-5 and 10.

At operation 810, an electronic device, such as one or more of the measurement devices 103-503, 1003 of FIGS. 1-5 and 10, may receive a measurement identifier, such as a QR code. At operation 820, the electronic device may obtain a measurement corresponding to the measurement identifier. At operation 830, the electronic device may register the measurement with a screening record associated with the measurement identifier.

In various examples, this example method 800 may be implemented as a group of interrelated software modules or components that perform various functions discussed herein. These software modules or components may be executed within a cloud network and/or by one or more computing devices, such as one or more of the measurement devices 103-503, 1003 of FIGS. 1-5 and 10.

Although the example method 800 is illustrated and described as including particular operations performed in a particular order, it is understood that this is an example. In various implementations, various orders of the same, similar, and/or different operations may be performed without departing from the scope of the present disclosure.

For example, the method 800 is illustrated and described as receiving a measurement identifier and obtaining and registering a measurement associated therewith. However, it is understood that this is an example. In other implementations, the electronic device may interact with an identity system to identify a user associated with the measurement and the electronic device may use such information to register the measurement with a corresponding screening record without using a measurement identifier. Various configurations are possible and contemplated without departing from the scope of the present disclosure.

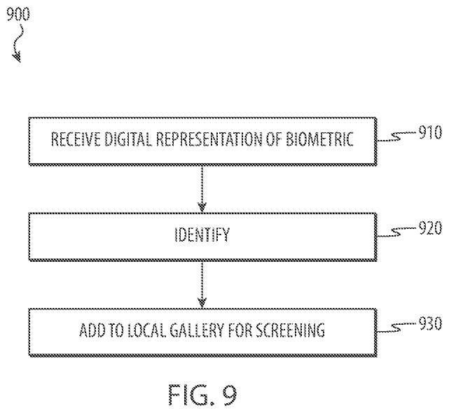

FIG. 9 is a flow chart illustrating a fourth example method 900 for medical screening entry. This method 900 may be performed by one or more of the systems 100-500 and 1000 of FIGS. 1-5 and 10.

At operation 910, an electronic device, such as one or more of the identity systems 206-506 of FIGS. 2-5, may receive one or more digital representations of one or more biometrics for one or more people. At operation 920, the electronic device may use the digital representations of the biometric to identify the person. At operation 930, the electronic device may add information for the person to a local gallery for screening, such as a local gallery used by a measurement device associated with a screening request corresponding to the person.

In various examples, this example method 900 may be implemented as a group of interrelated software modules or components that perform various functions discussed herein. These software modules or components may be executed within a cloud network and/or by one or more computing devices, such as one or more of the identity systems 206-506 of FIGS. 2-5.

Although the example method 900 is illustrated and described as including particular operations performed in a particular order, it is understood that this is an example. In various implementations, various orders of the same, similar, and/or different operations may be performed without departing from the scope of the present disclosure.

For example, operation 930 is illustrated and described as adding information for the person to a local gallery. However, it is understood that this is an example. In various implementations, operation 930 may be omitted and the method 900 may be used to identify the person using the digital representation of the biometric. Various configurations are possible and contemplated without departing from the scope of the present disclosure.

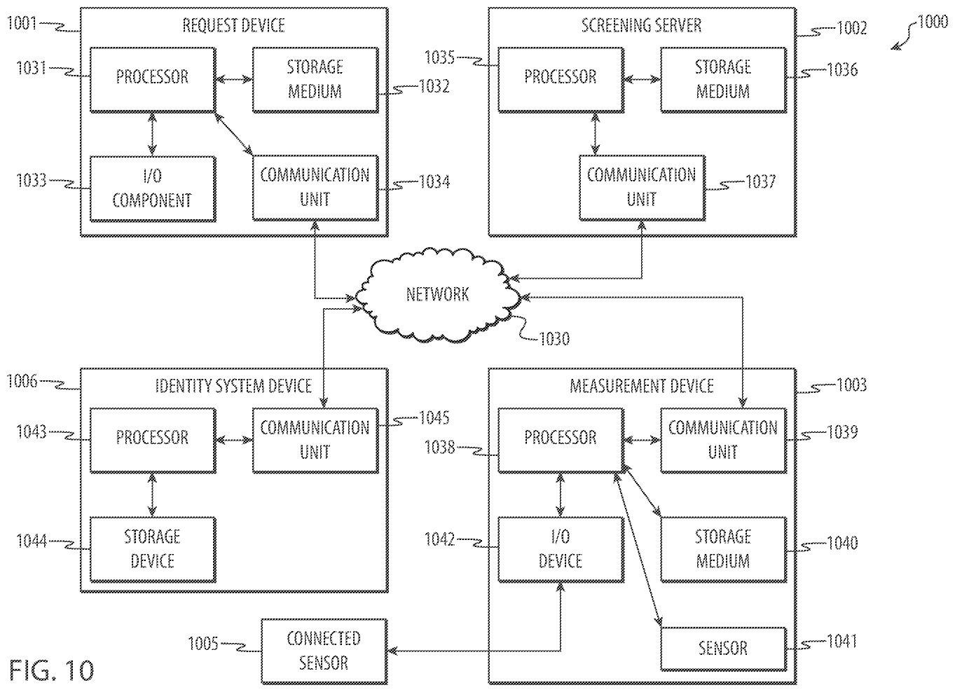

FIG. 10 illustrates example relationships among example components 1000 that may be used to implement one or more of the systems of FIGS. 1-5. One or more request devices 1001 and one or more measurement devices 1003 may communicate with one or more screening servers 1002 via one or more networks 1030. In some examples, or more identity system devices 1006 may also be able to communicate with one or more of the request device 1001, the screening server 1002, and/or the measurement device 1003 via the one or more networks 1030.

The identity system device 1006 may store identity information (such as one or more names, addresses, telephone numbers, social security numbers, patient identification numbers or other identifiers, insurance data, financial data, health information (such as one or more temperatures, pupil dilation, medical diagnoses, immunocompromised conditions, medical histories, medical records, infection statuses, vaccinations, immunology data, results of antibody tests evidencing that a person has had a particular communicable illness and recovered, blood test results, saliva test results, and/or the like), and so on) associated with the identities of people (which may be verified identities, where the identities are verified as corresponding to the particular person named and/or where the identity information is verified as valid). Alternatively and/or additionally, some or all of the health information may be stored separately from the identity information but otherwise associated with the identity information, such as in a HIPAA compliant or other data store or enclave and/or a blockchain and/or other auditable record or ledger, such as via one or more identifiers for such health information that may be stored in and/or otherwise associated with the identity information. Such a data store or enclave may be stored on one or more different storage media than the identity information, or may be stored on the same storage medium or media and logically isolated from the identity information. The health information may be simultaneously and/or substantially simultaneously accessible as the identity information, such as where the identity information includes a health information identifier or key that may be used to access the separately stored health information. The identity system device 1006 may control access to the identity information and/or the health information using identification information that is associated with the identity information. The identification information may include biometric data (which may include one or more digital representations of one or more fingerprints, blood vessel scans, palm-vein scans, voiceprints, facial images, retina images, iris images, deoxyribonucleic acid sequences, heart rhythms, gaits, and so on), one or more logins and/or passwords, authorization tokens, social media and/or other accounts, and so on. In various implementations, the identity system device 1006 may allow the person associated with an identity to control access to the identity information, the health information, and/or other information (such as payment account information, health information (such as medical records, HIPAA protected information in order to be compliant with various legal restrictions, and so on), contact information, and so on. The identity system device 1006 may control access to such information according to input received from the person. The identity system device 1006 may be operable to communicate with one or more electronic devices (such as the request device 1001, the screening server 1002, the measurement device 1003, and so on) in order to handle requests to provide the identity information and/or the health information, update and/or otherwise add to the identity information and/or the health information, provide attestations regarding and/or related to the identity information and/or the health information (such as whether or not a person is of a particular age, whether or not a person has a particular license or insurance policy, whether or not a person has been monitored as having particular health information, whether or not a person has had a particular vaccination, whether or not an antibody test evidences that a person has had a particular communicable illness and recovered, whether or not a person has a particular ticket or authorization, whether or not a person has been monitored as having particular antibodies, whether or not a person has been assigned a particular medical diagnosis, and so on), evaluate health information stored in the identity information and/or otherwise associated with the identity information and/or other information stored in the identity information, perform transactions, allow or deny access, route one or more persons, and/or perform one or more other actions.

The identity system device 1006 may be any kind of electronic device and/or cloud and/or other computing arrangement. Examples of such devices include, but are not limited to, one or more desktop computing devices, laptop computing devices, mobile computing devices, wearable devices, tablet computing devices, mobile telephones, smart phones, printers, displays, vehicles, kitchen appliances, entertainment system devices, digital media players, and so on. The identity system device 1006 may include one or more processors 1043 and/or other processing units or controllers, communication units 1045, non-transitory storage media 1044 (which may take the form of, but is not limited to, a magnetic storage medium; optical storage medium; magneto-optical storage medium; read only memory; random access memory; erasable programmable memory; flash memory; and so on), and/or other components. The processor 1043 may execute one or more sets of instructions stored in the non-transitory storage media 1044 to perform various functions, such as communicating via the communication unit 1045 and so on.

Similarly, the request device 1001 may be any kind of device. The request device 1001 may include one or more processors 1031 and/or other processing units and/or controllers, one or more non-transitory storage media 1032, communication units 1034, input and/or output components 1033 (such as one or more keyboards, computer mice, touch screens, microphones, displays, speakers, health sensors (such as a thermometer and/or other thermal sensor and/or thermal camera and/or a thermal imaging sensor, a blood pressure sensor, a blood test sensor, a blood vessel scanner, a palm-vein scanner, a still image and/or video camera, a 2D and/or 3D image sensor, a saliva sensor, a breath sensor, a deoxyribonucleic acid sensor, a heart rhythm monitor, a microphone, sweat sensors, and so on), biometric readers (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on), and so on. The processor 1031 may execute one or more sets of instructions stored in the non-transitory storage media 1032 to perform various functions, such as communicating via the communication unit 1034 and so on.

Likewise, the screening server 1002 may be any kind of device. The screening server 1002 may include one or more processors 1035 and/or other processing units and/or controllers, one or more non-transitory storage media 1036, communication units 1037, and so on. The processor 1035 may execute one or more sets of instructions stored in the non-transitory storage media 1036 to perform various functions, such as communicating via the communication unit 1037 and so on.

Similarly, the measurement device 1003 may be any kind of device. The measurement device 1003 may include one or more processors 1038 and/or other processing units and/or controllers, one or more non-transitory storage media 1040, communication units 1039, input and/or output devices 1042 (such as one or more keyboards, computer mice, touch screens, microphones, displays, speakers, health sensors (such as a thermometer and/or other thermal sensor and/or thermal camera and/or a thermal imaging sensor, a blood pressure sensor, a blood test sensor, a blood vessel scanner, a palm-vein scanner, a still image and/or video camera, a 2D and/or 3D image sensor, a saliva sensor, breath sensor, a deoxyribonucleic acid sensor, a heart rhythm monitor, a microphone, sweat sensors, and so on), biometric readers (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on), sensors (such as a thermometer and/or other thermal sensor and/or thermal camera and/or thermal imaging sensor, a blood pressure sensor, a blood test sensor, a blood vessel scanner, a palm-vein scanner, a still image and/or video camera, a 2D and/or 3D image sensor, a saliva sensor, a breath sensor, a deoxyribonucleic acid sensor, a heart rhythm monitor, a microphone, sweat sensors, and so on), and so on. The processor 1038 may execute one or more sets of instructions stored in the non-transitory storage media 1040 to perform various functions, such as communicating via the communication unit 1039 and so on. In some implementations, the input and/or output devices 1042 may include a connection for communicating with a connected sensor 1005 (such as a thermometer and/or other thermal sensor and/or a thermal camera and/or thermal imaging sensor, a blood pressure sensor, a blood test sensor, a blood vessel scanner, a palm-vein scanner, a still image and/or video camera, a 2D and/or 3D image sensor, a saliva sensor, a breath sensor, a deoxyribonucleic acid sensor, a heart rhythm monitor, a microphone, sweat sensors, and so on) or other interface.

Although FIG. 10 is illustrated and described as including particular components arranged in a particular configuration that perform particular functions, it is understood that this is an example. In various implementations, various arrangements of various components that perform various functions may be implemented without departing from the scope of the present disclosure.

For example, in some examples, the identity system device 1006 and/or the connected sensor 1005 may be omitted. Various configurations are possible and contemplated without departing from the scope of the present disclosure.

Measurements obtained from a person (such as a user) and/or any kind of test results (such as antibody test results, a temperature, a white blood cell count, a thermal scan, a blood test, a saliva test, a breath test, and so) and/or other health information may be associated with identity information for a person in a variety of different ways. For example, a person may indicate that a test result is his and request association. By way of another example, an entity associated with the test results (such as a testing facility, a provider of a home test kit, a pharmacy, and so on) may communicate the test results and the test results may then be associated with identity information for a person. Regardless how requests for association of such test results and/or other health information may be initiated, one or more electronic devices receiving such a request may verify correspondence between the test results and one or more identities before associating the test results with one or more sets of identity information. This verification may be performed in a variety of different ways.

By way of example, a person may take a rapid result blood test at a kiosk at a drug store. The kiosk may include a fingerprint sensor that obtains an image of the person's fingerprint at the same time that the kiosk pricks the person's finger to obtain a blood sample to test, which may ensure that the blood is from the same person as the fingerprint image. The person may be identified using the fingerprint image and test results of the rapid result blood test may be associated with the identity of the person. Various configurations are possible and contemplated without departing from the scope of the present disclosure.

In another example, the person may take a test at a kiosk that may be video monitored. The person may be identified from the video using facial recognition techniques and test results (whether communicated by the kiosk, monitored on the video, and so on) may be associated accordingly. Various configurations are possible and contemplated without departing from the scope of the present disclosure.

By way of another example, a person may take a test at a kiosk and use an app for an identity system executing on a mobile device to monitor the test, provide one or more digital representations of biometrics in order to identify himself, and so on. Various configurations are possible and contemplated without departing from the scope of the present disclosure.

In another example, a person may obtain a home test for a communicable illness. The person may complete the home test and self-enter the test results into an app for an identity system executing on a mobile device. The app may obtain one or more digital representations of one or more biometrics from the person in order to determine an identity for the person and associate the test results with the identity.

In some examples, a trustworthiness score may be determined for a person and/or used in determining whether or not to allow the person access and/or as part of making other determinations. Such a trust score may be based on publicly available financial and/or other information that indicates a general trustworthiness of the person, behavior patterns that tend to indicate a general trustworthiness of the person, watch lists, criminal behavior and/or civil wrongdoing, and so on. Such a trustworthiness score may also be based on other information, such as whether or not a person has ever provided false or misleading health information, whether or not a person has ever withheld information regarding a health risk, whether or not a person has ever asserted health and later been found to be ill (such as receiving medical services to treat a communicable illness shortly after asserting that they had not been exposed to the communicable illness and so on), whether or not the person or a connected person is diagnosed with a communicable illness after voluntarily attending a situation where people with a risk of the communicable illness were not supposed to attend, and so on. Such a trustworthiness score may be used in a variety of different ways. For example, the test result self-enter example above may be restricted to people with trustworthiness scores above a threshold, whereas people with trustworthiness scores at or below the threshold may be required to validate the test results in some way. Various configurations are possible and contemplated without departing from the scope of the present disclosure.

For example, the app may be used to capture an image and/or other proof of the test result of the home test, which may include obtaining a time stamp and/or other test proof. By way of another example, the app may be used to capture video of the person taking the home test as well as the test result to ensure that the person who provides the digital representation of the biometric is the same person who took the test. Various configurations are possible and contemplated without departing from the scope of the present disclosure.

In another example, the home test may be registered to the identity of the person. By way of illustration, the app may be used to scan a QR code or other bar code on the home test and/or serial number or other identifier on the test as well as one or more digital representations of one or more biometrics for the person. This may be used to associate the particular home test with the identity of the person, which may deter the person from having another person take the test and/or falsely reporting the test result. By way of another illustration, the home test may be associated with the identity before being provided to the person, such as where the person has been prompted to take the home test and is provided a home test already registered to his identity. In some examples, the home test may auto report results and thus the auto reported test results may be associated with the identity to whom the test is registered. In various examples, the app may be used to monitor the person taking the test to ensure that the identity to whom the home test is registered corresponds to the person who takes the test. Various configurations are possible and contemplated without departing from the scope of the present disclosure.

In some example implementations where trustworthiness scores may be determined, people who cannot be determined to be trustworthy may not be allowed to take home tests and may instead have to take monitored tests. In other example implementations, less trustworthy people may be required to submit to higher levels of verification for home tests, such as video monitoring during testing, whereas trustworthy people may be allowed to self-enter test results. The degree of certainty associated with the test result may be associated with the trustworthiness score of the person, the verifications associated with the home test, and so on. In some examples, the degree of certainty associated with the test result may be evaluated as part of evaluating the health information, such as allowing access if the degree of certainty is at least a threshold and denying otherwise, and so on. Various configurations are possible and contemplated without departing from the scope of the present disclosure.

In various examples, the test results may be digitally notarized by the person and/or another person. For example, the person and/or another person may provide one or more digital representations of one or more biometrics when test results are provided in order for the person and/or the other person to attest that the provided test results are accurate. In some situations, a trustworthy person may attest to the test results for a less trustworthy person so that the less trustworthy person may use the home test, may self-enter the test result, and so on. By way of illustration, the other person may be a pharmacist, a doctor, a nurse, and so on. Various configurations are possible and contemplated without departing from the scope of the present disclosure.

Although the above describes a home test, it is understood that this is an example. In other examples, the above techniques may be used for a variety of administered tests and/or other kinds of tests without departing from the scope of the present disclosure. Various configurations are possible and contemplated without departing from the scope of the present disclosure.

In some implementations, multiple test results may be obtained and/or associated with a person's identity. For example, some antibody tests may have a high degree of false negative results (such as 20%, 30%, or even higher) such that the antibody tests may need to be repeated multiple times to verify that a person has previously overcome a particular communicable illness. As such, multiple test results may be obtained and associated with the identity and a later positive test result indicating that the person has previously overcome a particular communicable illness may override a previous negative test result that falsely indicated that the person had not previously overcome the particular communicable illness. By way of illustration, the person may know and/or suspect that he has already had and overcome the particular communicable illness. The person may obtain and take a home test, which may not prove that the person has overcome the particular illness. The person may then obtain another home test and retake until proof is obtained. Various configurations are possible and contemplated without departing from the scope of the present disclosure.

In other implementations, a person providing multiple test results may indicate that the person is trying to obtain a falsely positive determination that the person desires instead of ameliorating a falsely negative determination. For example, instead of an antibody test that never indicates that a person has overcome an illness that the person has not but sometimes indicates that the person has not overcome an illness that the person has, the reverse may be true. As such, the person may repeat the test hoping to obtain a clearance that the person does not deserve. In such a case, multiple test results may be tracked to determine that the person is trying to game the tests and override a clearance that might otherwise be provided. In some implementations of such examples, a trustworthiness score of the person may be evaluated to determine when a person may be attempting to game the tests and/or when the person may be appropriately pursuing multiple tests for certainty. Various configurations are possible and contemplated without departing from the scope of the present disclosure.

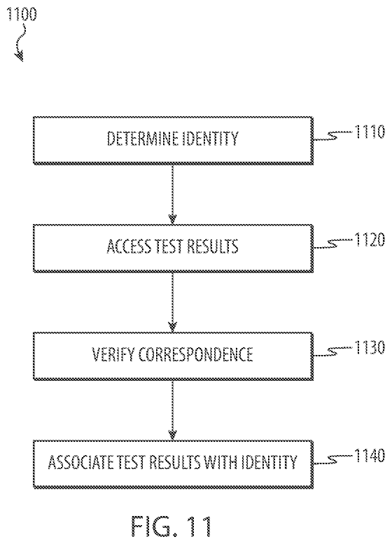

FIG. 11 is a flow chart illustrating a fifth example method 1100 for medical screening entry. This method 1100 may be performed by one or more of the systems 100-500 and 1000 of FIGS. 1-5 and 10.

At operation 1110, an electronic device, such as a component of one or more of the identity systems 206-506 of FIGS. 2-5 and/or the identity system device 1006 of FIG. 10, may determine an identity of a person. For example, the electronic device may compare a digital representation of a biometric for the person with stored biometric data associated with identity information in order to determine the identity of the person. At operation 1120, the electronic device may access test results. The electronic device may access the test results in a variety of different ways. In some implementations, the electronic device may receive the test results from an entity associated with the test results (such as a testing facility, a provider of a home test kit, a pharmacy, and so on). In other implementations, the electronic device may receive information from the person that the electronic device may use to look up and/or otherwise access the test results (such as a test identifier and so on). In still other implementations, the electronic device may receive the test results from the person. In such an implementation, the electronic device may then communicate an entity associated with the test results (such as a testing facility, a provider of a home test kit, a pharmacy, and so on) to verify that the received test results are valid. In some examples, the test results may be machine readable, encrypted, and/or otherwise stored in such a way that the test results are not readable by the person and must be accessed via the electronic device or other authorized device in order for the test results to be comprehensible. This may prevent the person from learning of adverse test results and then not associating those adverse test results with his identity information, particularly when the test results are obtained from a home test as opposed to a monitored test, which a person may be more able to avoid reporting if the person knew of an adverse result before reporting of the result.

At operation 1130, the electronic device may verify correspondence between the test results and the identity. In some implementations, this may involve comparing biographic and/or other information stored in and/or otherwise associated with identity information for the person with biographic and/or other information associated with the test results. For example, such information may include first name, middle name or initial, last name, insurance information, address, gender, social security number and/or other identifier, and so on. When the electronic device determines that the biographic and/or other information stored in and/or otherwise associated with identity information for the person corresponds to that from the test results, the electronic device may determine that the test results are for the person and determine that the correspondence between the test results and the identity is verified. In some examples, the electronic device may determine that one or more items of the biographic and/or other information stored in and/or otherwise associated with identity information for the person matches that from the test results in order to verify correspondence between the test results and the identity. In other examples, the electronic device may determine that a threshold certainty level is met based on similarity between one or more items of the biographic and/or other information stored in and/or otherwise associated with identity information for the person and that from the test results in order to verify correspondence between the test results and the identity (such as where one lists a full middle name and the other lists a middle initial, where one lists a current address and the other lists an old address, and so on). In other implementations, the electronic device may verify correspondence by verifying that the test results are not associated with health information that is inconsistent with health information stored in and/or otherwise associated with the identity information. For example, the test results may include data regarding age of the test subject, blood type of the test subject, DNA of the test subject, gender of the test subject, and/or other health information about the test subject that may have been obtained from a sample related to the test (such as a blood sample, a DNA sample, a mucus sample, and so on) and such data may be compared to health information stored in and/or otherwise associated with the identity information. By way of illustration, the electronic device may verify correspondence by ensuring that the test results are not associated with an age inconsistent with that of the person, blood type inconsistent with that of the person, DNA inconsistent with that of the person, gender inconsistent with that of the person, and/or other health information inconsistent with that of the person. Such data may be gathered during testing. In implementations where the test is a home test kit, such data may be gathered by collecting test kits after test completion and later testing samples included in the collected test kits in order to obtain the data, whether routinely and/or in situations where additional verification is determined to be performed. In still other implementations, identities of people may be biometrically determined upon entry to a testing facility and tests results may be verified as corresponding to those identities based upon the identities biometrically determined upon entry. Various configurations are possible and contemplated without departing from the scope of the present disclosure.

At operation 1140, after verifying the correspondence, the electronic device may associate the test results with the identity. This may include storing the test results in the identity information, storing the test results in a data store or enclave and/or a blockchain and/or other auditable record or ledger associated with the identity, and so on. For example, a blockchain and/or other auditable record or ledger may include one or more data blocks with one or more test result identifiers and/or one or more identifiers for and/or associated with the identity. The one or more test result identifiers and/or one or more identifiers for and/or associated with the identity may be used to associate the test results with identity information for the person, such as where the one or more test result identifiers and/or one or more identifiers for and/or associated with the identity are stored in the identity information and used to access the test results from the blockchain and/or other auditable record or ledger.

In various examples, this example method 900 may be implemented as a group of interrelated software modules or components that perform various functions discussed herein. These software modules or components may be executed within a cloud network and/or by one or more computing devices, such as a component of one or more of the identity systems 206-506 of FIGS. 2-5, one or more of the request devices 101-501, 1001 of FIGS. 1-5 and 10, and/or the identity system device 1006 of FIG. 10.

Although the example method 1100 is illustrated and described as including particular operations performed in a particular order, it is understood that this is an example. In various implementations, various orders of the same, similar, and/or different operations may be performed without departing from the scope of the present disclosure.

For example, the method 1100 illustrates and describes verifying correspondence between accessed test results and a determined identity. However, it is understood that this is an example. In other implementations, correspondence between a test and an identity may instead be verified, whether prior to, subsequent to, and/or during determination of the test results. For example, a home test kit may be registered to an identity when purchased and correspondence between the home test kit and the identity may be verified. An identity (and/or family, household, and/or other relationship associated with an identity) may be only able to be associated with the purchase of one home test kit in order to prevent and/or reduce the possibility that a person will obtain multiple test kits and report that person's results for multiple different people (and/or otherwise reduce the incentive for the person to attempt to invalidly associate a test result with his and/or another person's identity). Exceptions may be made for situations where a test has a high false negative rate, such as where a test with a high false negative rate may be overridden by a later test with sufficient verification that both tests were taken by the same person, where a less accurate test may be overridden by a later and more accurate test, where a home test may be overridden by a monitored test, where multiple tests are taken into account when determining a confidence level regarding whether the multiple tests were taken by the same person or multiple people, where a person's status may have changed between a first test and a second test (such as where the person had not acquired antibodies for a communicable illness before the first test but did before the second test), where a time threshold (such as a week) has passed between multiple tests, and so on. In various implementations, test kits registered to an identity may be required to be used within a time period, such as within three days of being registered, in order to prevent and/or reduce the possibility that people may register tests and then provide those tests to other people (and/or otherwise reduce the incentive for the person to attempt to invalidly associate a test result with his and/or another person's identity). Various configurations are possible and contemplated without departing from the scope of the present disclosure.

Other techniques may be used to prevent and/or reduce the possibility that a person will obtain multiple test kits and report that person's results for multiple different people (and/or otherwise reduce the incentive for the person to attempt to invalidly associate a test result with his and/or another person's identity). For example, test results may not be provided to a person until all tests for a family, household, and/or other relationship associated with the person's identity have been completed. In other examples, tests result may be machine readable, encrypted, and/or otherwise stored in such a way that the test results are not readable by the person and must be accessed via the electronic device and/or other authorized device after reporting in order for the test results to be comprehensible.

In still other examples, correspondence between a test and an identity may instead be verified by obtaining video, images, and/or other data monitoring the person taking the test, such as via a home test kit. Such video, images, and/or other data may be collected and stored as proof of the correspondence, analyzed by one or more electronic devices and/or human monitors to look for suspicious activity and/or identify the test and/or the person (such as where a QR code and/or other identifier is captured from the test; where facial and/or other biometric recognition is used to identify the person in the video, images, and/or other data; where a remote witness attests that he witnessed the person take the test; and so on). Such video, images, and/or other data may be analyzed to verify chain of custody of the test. In other words, the video, images, and/or other data may be analyzed to verify that the same person unsealed and/or opened the test, performed the test on himself, sealed and/or otherwise completed the test, and provided the sealed and/or otherwise completed test sample for testing evaluation.

In yet other examples, correspondence between a test and an identity may instead be verified using an attestation of another person who witnessed the test, whether in person or remotely. For example, various people may be designated as authorized to witness tests. In another example, people may be authorized to witness tests upon completion of one or more background checks. In still other examples, lab and/or testing and/or medical service provider personnel may remotely witness people taking home tests. Various configurations are possible and contemplated without departing from the scope of the present disclosure.

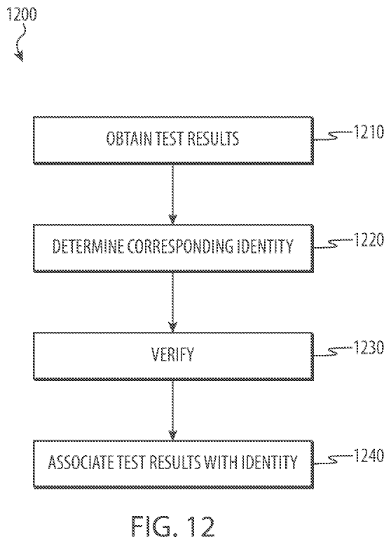

FIG. 12 is a flow chart illustrating a sixth example method 1200 for medical screening entry. This method 1200 may be performed by one or more of the systems 100-500 and 1000 of FIGS. 1-5 and 10.

At operation 1210, an electronic device, such as a component of one or more of the identity systems 206-506 of FIGS. 2-5 and/or the identity system device 1006 of FIG. 10, may obtain test results. At operation 1220, the electronic device may determine the corresponding identity of a person. For example, the electronic device may compare a digital representation of a biometric for the person with stored biometric data associated with identity information in order to determine the identity of the person that purportedly corresponds to the test results.

At operation 1230, the electronic device may verify correspondence of the test results to the identity. In other words, the electronic device may verify that the identity of the person that purportedly corresponds to the test results actually does correspond to the test results.

By way of illustration, the electronic device may compare biographic and/or other information stored in and/or otherwise associated with identity information for the person with biographic and/or other information associated with the test results. For example, such information may include first name, middle name or initial, last name, insurance information, address, gender, social security number and/or other identifier, and so on. When the electronic device determines that the biographic and/or other information stored in and/or otherwise associated with identity information for the person corresponds to that from the test results, the electronic device may determine that the test results are for the person and determine that the correspondence between the test results and the identity is verified.

In some implementations, the electronic device may determine that one or more items of the biographic and/or other information stored in and/or otherwise associated with identity information for the person matches that from the test results in order to verify correspondence between the test results and the identity. In other implementations, the electronic device may determine that a threshold certainty level is met based on similarity between one or more items of the biographic and/or other information stored in and/or otherwise associated with identity information for the person and that from the test results in order to verify correspondence between the test results and the identity (such as where one lists a full middle name and the other lists a middle initial, where one lists a current address and the other lists an old address, and so on).

By way of another illustration, the electronic device may verify correspondence by verifying that the test results are not associated with health information that is inconsistent with health information stored in and/or otherwise associated with the identity information. For example, the test results may include data regarding age of the test subject, blood type of the test subject, DNA of the test subject, gender of the test subject, and/or other health information about the test subject that may have been obtained from a sample related to the test (such as a blood sample, a DNA sample, a mucus sample, and so on) and such data may be compared to health information stored in and/or otherwise associated with the identity information. In some implementations, the electronic device may verify correspondence by ensuring that the test results are not associated with an age inconsistent with that of the person, blood type inconsistent with that of the person, DNA inconsistent with that of the person, gender inconsistent with that of the person, and/or other health information inconsistent with that of the person. Such data may be gathered during testing. In implementations where the test is a home test kit, such data may be gathered by collecting test kits after test completion and later testing samples included in the collected test kits in order to obtain the data, whether routinely and/or in situations where additional verification is determined to be performed.

In yet another illustration, identities of people may be biometrically determined upon entry to a testing facility. In such a configuration, test results may be verified as corresponding to identities based upon the identities biometrically determined upon entry. Various configurations are possible and contemplated without departing from the scope of the present disclosure.

At operation 1240, the electronic device may associate the test results with the identity. This may include storing the test results in the identity information, storing the test results in a data store or enclave and/or a blockchain and/or other auditable record or ledger associated with the identity, and so on. For example, a blockchain and/or other auditable record or ledger may include one or more data blocks with one or more test result identifiers and/or one or more identifiers for and/or associated with the identity. The one or more test result identifiers and/or one or more identifiers for and/or associated with the identity may be used to associate the test results with identity information for the person, such as where the one or more test result identifiers and/or one or more identifiers for and/or associated with the identity are stored in the identity information and used to access the test results from the blockchain and/or other auditable record or ledger.

In various examples, this example method 1200 may be implemented as a group of interrelated software modules or components that perform various functions discussed herein. These software modules or components may be executed within a cloud network and/or by one or more computing devices, such as a component of one or more of the identity systems 206-506 of FIGS. 2-5 and/or the identity system device 1006 of FIG. 10.

Although the example method 1200 is illustrated and described as including particular operations performed in a particular order, it is understood that this is an example. In various implementations, various orders of the same, similar, and/or different operations may be performed without departing from the scope of the present disclosure.

For example, the method 1200 illustrates obtaining the test results and determining the identity as separate, sequential operations. However, it is understood that this is an example. In other implementations, such operations may be performed in any order, including simultaneously, concurrently, and/or substantially simultaneously and/or concurrently. Various configurations are possible and contemplated without departing from the scope of the present disclosure.

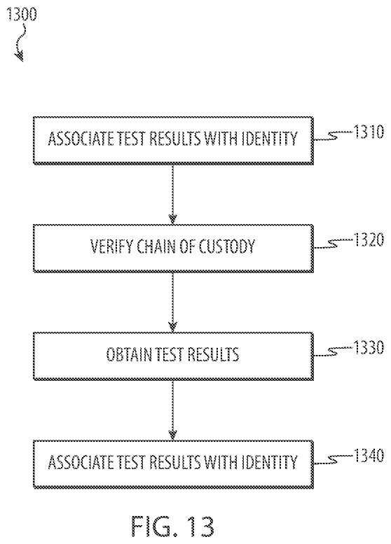

FIG. 13 is a flow chart illustrating a seventh example method 1300 for medical screening entry. This method 1300 may be performed by one or more of the systems 100-500 and 1000 of FIGS. 1-5 and 10.

At operation 1310, an electronic device, such as a component of one or more of the identity systems 206-506 of FIGS. 2-5 and/or the identity system device 1006 of FIG. 10, may associate a test with an identity. For example, a home test kit may be registered to an identity when purchased. By way of another example, an app or similar mechanism may be used to scan a QR code or other bar code and/or identifier on the home test and/or serial number or other identifier on the test as well as one or more digital representations of one or more biometrics for the person, associating the particular home test with the identity of the person, which may deter the person from having another person take the test and/or falsely reporting the test result. In still other examples, a person has been prompted to take a home test and may be provided a home test already registered to his identity. An identity (and/or family, household, and/or other relationship associated with an identity) may be only able to be associated with the purchase of one home test kit in order to prevent and/or reduce the possibility that a person will obtain multiple test kits and report that person's results for multiple different people (and/or otherwise reduce the incentive for the person to attempt to invalidly associate a test result with his and/or another person's identity). Exceptions may be made for situations where a test has a high false negative rate, such as where a test with a high false negative rate may be overridden by a later test with sufficient verification that both tests were taken by the same person, where a less accurate test may be overridden by a later and more accurate test, where a home test may be overridden by a monitored test, where multiple tests are taken into account when determining a confidence level regarding whether the multiple tests were taken by the same person or multiple people, where a person's status may have changed between a first test and a second test (such as where the person had not acquired antibodies for a communicable illness before the first test but did before the second test), where a time threshold (such as a week) has passed between multiple tests, and so on. In various implementations, test kits registered to an identity may be required to be used within a time period, such as within three days of being registered, in order to prevent and/or reduce the possibility that people may register tests and then provide those tests to other people (and/or otherwise reduce the incentive for the person to attempt to invalidly associate a test result with his and/or another person's identity). Various configurations are possible and contemplated without departing from the scope of the present disclosure.

At operation 1320, the electronic device may verify chain of custody of the test. For example, chain of custody of the test may be verified by obtaining video, images, and/or other data monitoring the person taking the test, which may a home test kit. Such video, images, and/or other data may be collected and stored as proof of the chain of custody of the test, analyzed by one or more electronic devices and/or human monitors to look for suspicious activity and/or identify the test and/or the person (such as where a QR code and/or other identifier is captured from the test; where facial and/or other biometric recognition is used to identify the person in the video, images, and/or other data; where a remote witness attests that he witnessed the person take the test; and so on). Such video, images, and/or other data may be analyzed to verify that the same person unsealed and/or opened the test, performed the test on himself, sealed and/or otherwise completed the test, and provided the sealed and/or otherwise completed test sample for testing evaluation.

Alternatively and/or additionally, chain of custody of the test may be verified using an attestation of another person who witnessed the test, whether in person or remotely. For example, various people may be designated as authorized to witness tests. In another example, people may be authorized to witness tests upon completion of one or more background checks. In still other examples, lab and/or testing and/or medical service provider personnel may remotely witness people taking home tests. Various configurations are possible and contemplated without departing from the scope of the present disclosure.

At operation 1330, the electronic device may obtain test results for the test. At operation 1340, the electronic device may associate the test results with the identity. This may include storing the test results in the identity information, storing the test results in a data store or enclave and/or a blockchain and/or other auditable record or ledger associated with the identity, and so on. For example, a blockchain and/or other auditable record or ledger may include one or more data blocks with one or more test result identifiers and/or one or more identifiers for and/or associated with the identity. The one or more test result identifiers and/or one or more identifiers for and/or associated with the identity may be used to associate the test results with identity information for the person, such as where the one or more test result identifiers and/or one or more identifiers for and/or associated with the identity are stored in the identity information and used to access the test results from the blockchain and/or other auditable record or ledger. In some implementations, the test may auto report results and thus the auto reported test results may be associated with the identity to whom the test is registered.

In various examples, this example method 1300 may be implemented as a group of interrelated software modules or components that perform various functions discussed herein. These software modules or components may be executed within a cloud network and/or by one or more computing devices, such as a component of one or more of the identity systems 206-506 of FIGS. 2-5 and/or the identity system device 1006 of FIG. 10.

Although the example method 1300 is illustrated and described as including particular operations performed in a particular order, it is understood that this is an example. In various implementations, various orders of the same, similar, and/or different operations may be performed without departing from the scope of the present disclosure.

For example, the method 1300 is illustrated and described as associating a test with an identity, verifying the chain of custody of the test, obtaining the test results, and associating the test results with the identity as separate, sequential operations. However, it is understood that this is an example. In other implementations, such operations may be performed in any order, including simultaneously, concurrently, and/or substantially simultaneously and/or concurrently. Various configurations are possible and contemplated without departing from the scope of the present disclosure.

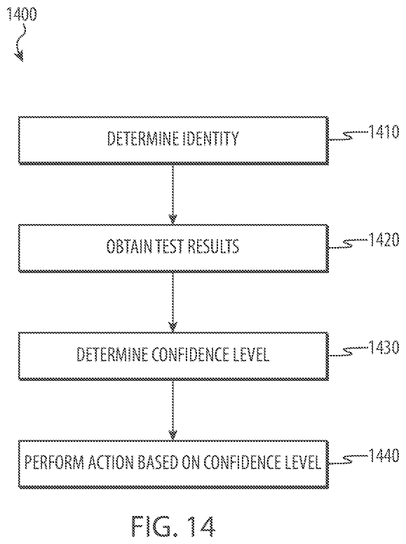

FIG. 14 is a flow chart illustrating an eighth example method 1400 for medical screening entry. This method 1400 may be performed by one or more of the systems 100-500 and 1000 of FIGS. 1-5 and 10.

At operation 1410, an electronic device, such as a component of one or more of the identity systems 206-506 of FIGS. 2-5 and/or the identity system device 1006 of FIG. 10, may determine an identity of a person. For example, the electronic device may compare a digital representation of a biometric for the person with stored biometric data associated with identity information in order to determine the identity of the person. At operation 1420, the electronic device may obtain test results. At operation 1430, the electronic device may determine a confidence level in the test results. At operation 1440, the electronic device may perform an action based on the confidence level. Such an action may include providing one or more attestations regarding the confidence level, controlling access based on the confidence level, allowing access when the confidence level is above a threshold, denying access when the confidence level is below the threshold, providing different levels of access for different confidence levels, storing the confidence level, providing information on how to change the confidence level, and so on.

For example, the confidence level may be determined based on whether the test is a home test kit, whether the test is a monitored test administered by medical personnel, whether or not the test is a home test registered to the person, whether or not biographic and/or other information included in and/or otherwise associated with identity information for the person corresponds to biographic and/or other information from the test, the degree to which biographic and/or other information included in and/or otherwise associated with identity information for the person corresponds to biographic and/or other information from the test, whether or not the test was witnessed, whether or not the test was witnessed in person, whether or not the test was witnessed remotely, whether or not video and/or images and/or other data monitoring the test is logged, whether or not video and/or images and/or other data monitoring the test is verified, whether or not a biological sample from the test was deposited, whether or not a biological sample from the test was verified as corresponding to the person, whether or not the test results are inconsistent with other test results, how much verification of the test and/or of the person was performed, a trustworthiness score of the person, the accuracy of the test, and/or any other factor that may indicate how accurate the test results are. The confidence level may then be used to perform an action, such as allowing the person access to an area (such as to an airport, restaurant, gym, flight, and so on), conditional to use of protective gear (such as a mask) and temperature or other health information verification when the confidence level is a low level, allow access conditional to use of protective gear without a temperature or other health information verification when the confidence level is a middle level, and access without use of protective gear and/or temperature or other health information verification when the access is a high level. Various configurations are possible and contemplated without departing from the scope of the present disclosure.

In various examples, this example method 1400 may be implemented as a group of interrelated software modules or components that perform various functions discussed herein. These software modules or components may be executed within a cloud network and/or by one or more computing devices, such as a component of one or more of the identity systems 206-506 of FIGS. 2-5 and/or the identity system device 1006 of FIG. 10.

Although the example method 1400 is illustrated and described as including particular operations performed in a particular order, it is understood that this is an example. In various implementations, various orders of the same, similar, and/or different operations may be performed without departing from the scope of the present disclosure.

For example, the method 1400 is illustrated and described as obtaining the test results. However, it is understood that this is an example. In some implementations, the method 1400 may be used to determine a confidence level in one or more test results and perform one or more actions based thereon without obtaining the test results. Various configurations are possible and contemplated without departing from the scope of the present disclosure.

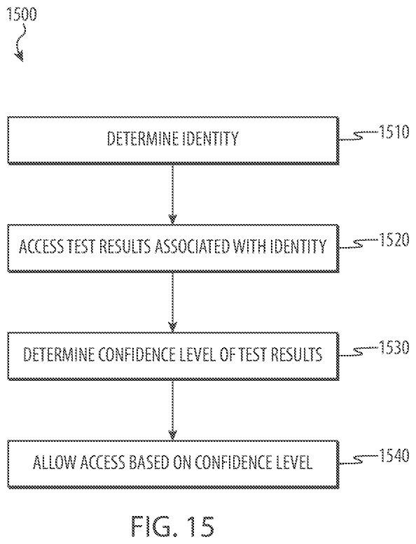

FIG. 15 is a flow chart illustrating a ninth example method 1500 for medical screening entry. This method 1500 may be performed by one or more of the systems 100-500 and 1000 of FIGS. 1-5 and 10.

At operation 1510, an electronic device, such as a component of one or more of the identity systems 206-506 of FIGS. 2-5 and/or the identity system device 1006 of FIG. 10, may determine an identity of a person. For example, the electronic device may compare a digital representation of a biometric for the person with stored biometric data associated with identity information in order to determine the identity of the person. At operation 1520, the electronic device may access test results associated with the identity. At operation 1530, the electronic device may obtain a confidence level in the test results.

For example, the electronic device may determine the confidence level based on whether the test is a home test kit, whether the test is a monitored test administered by medical personnel, whether or not the test is a home test registered to the person, whether or not biographic and/or other information included in and/or otherwise associated with identity information for the person corresponds to biographic and/or other information from the test, the degree to which biographic and/or other information included in and/or otherwise associated with identity information for the person corresponds to biographic and/or other information from the test, whether or not the test was witnessed, whether or not the test was witnessed in person, whether or not the test was witnessed remotely, whether or not video and/or images and/or other data monitoring the test is logged, whether or not video and/or images and/or other data monitoring the test is verified, whether or not a biological sample from the test was deposited, whether or not a biological sample from the test was verified as corresponding to the person, whether or not the test results are inconsistent with other test results, how much verification of the test and/or of the person was performed, a trustworthiness score of the person, the accuracy of the test, and/or any other factor that may indicate how accurate the test results are. The confidence level may be on a numeric and/or other scale, such as a scale involving levels 1 through 5 where 1 is the lowest and 5 is the highest.

At operation 1540, the electronic device may allow access based on the confidence level. For example, the electronic device may allow different types of access to an area, such as to an airport, restaurant, gym, flight, and so on, based upon the confidence level associated with the test. By way of illustration, the electronic device may determine that the confidence level is 0 for no test, 1 for a home test, 2 for a remotely monitored home test, 3 for a home test where video of test administration was stored, 4 for a home test where a biological sample from the test was provided and verified as corresponding to the person, and 5 for a professionally administered and monitored test. The electronic device may then deny the person access when the confidence level is 0, allow the person access conditional to use of protective gear (such as a mask) and temperature or other health information verification when the confidence level is 1, allow access other than to congregating areas conditional to use of protective gear without a temperature or other health information verification when the confidence level is 2, allow access other than to congregating areas without use of protective gear and/or temperature or other health information verification when the access is 3, allow access even to congregating areas conditional to use of protective gear without a temperature or other health information verification when the confidence level is 4, and allow access even to congregating areas without use of protective gear and/or temperature or other health information verification when the access is 5. Various configurations are possible and contemplated without departing from the scope of the present disclosure.

In various examples, this example method 1500 may be implemented as a group of interrelated software modules or components that perform various functions discussed herein. These software modules or components may be executed within a cloud network and/or by one or more computing devices, such as a component of one or more of the identity systems 206-506 of FIGS. 2-5 and/or the identity system device 1006 of FIG. 10.

Although the example method 1500 is illustrated and described as including particular operations performed in a particular order, it is understood that this is an example. In various implementations, various orders of the same, similar, and/or different operations may be performed without departing from the scope of the present disclosure.

For example, the method 1500 is illustrated and described as allowing access based on the confidence level. However, it is understood that this is an example. In other implementations, other actions may be performed. For example, in some implementations, access may be denied based on the confidence level. Various configurations are possible and contemplated without departing from the scope of the present disclosure.

In various examples of implementations that use techniques of the present disclosure, test results (such as results of an antibody test, a temperature test, and/or any other kind of medical and/or health test) may be recorded in a blockchain and/or other auditable record or ledger. The blockchain and/or other auditable record or ledger may include one or more test result identifiers and/or one or more identifiers for a person associated with the test result. The one or more test result identifiers and/or one or more identifiers for the person may be used to associate the test results with identity information for the person, such as where the one or more test result identifiers and/or one or more identifiers for the person are stored in the identity information and used to access the test results from the blockchain and/or other auditable record or ledger. In some examples, the test results in the blockchain and/or other auditable record or ledger may be machine readable, encrypted, and/or otherwise stored in such a way that the test results are not readable by the person from the blockchain and/or other auditable record or ledger and must be accessed via the identity information in order for the test results to be comprehensible. This may prevent a person from learning of adverse test results stored in the blockchain and/or other auditable record or ledger and then not associating those adverse test results with the identity information.

In various implementations, a system for medical screening entry may include an application operable to execute on a request device, a screening server, and a measurement device. The application may receive a request for screening from a person. The application may communicate with the screening server to perform a screening interaction. The application may receive a measurement identifier from the screening server. The application may provide the measurement identifier to the measurement device. The measurement device may obtain a measurement from the person. The measurement device may register the measurement with a screening record for the person. The application may receive a screening access identifier from the screening server. The application may provide the screening access identifier to an access control station.

In some examples, the measurement identifier may be a Quick Read code. In various examples, the screening access identifier may be a Quick Read code.

In some implementations, a system for medical screening entry may include an application operable to execute on a request device, a screening server, an identity system, and a measurement device. The application may receive a request for screening from a person. The application may communicate with the identity system to obtain an authentication for the person. The application may communicate with the screening server to perform a screening interaction wherein the application may provide the authentication to the screening server. The application may receive a measurement identifier from the screening server. The application may provide the measurement identifier to the measurement device. The measurement device may obtain a measurement from the person. The measurement device may register the measurement with a screening record for the person. The application may receive a screening access identifier from the screening server. The application may provide the screening access identifier to an access control station.

In various examples, personal health information may be omitted from the measurement identifier. In some examples, personal identification information may be omitted from the measurement identifier. In a number of examples, the measurement device may obtain the measurement using an integrated sensor. In various examples, the measurement device may obtain the measurement using a connected sensor. In some examples, the measurement device may obtain the measurement from a manual entry obtained from an operator via an interface.

In a number of implementations, a system for medical screening entry may include an application operable to execute on a request device, a screening server, an identity system, and a measurement device. The application may receive a request for screening from a person. The application may communicate with the identity system to obtain an authentication for the person and add information for the person to a local gallery. The application may communicate with the screening server to perform a screening interaction wherein the application provides the authentication to the screening server. The measurement device may use the local gallery to obtain an identification for the person. The measurement device may obtain a measurement from the person. The measurement device may register the measurement with a screening record for the person. The application may receive a screening access identifier from the screening server. The application may provide the screening access identifier to an access control station.

In some examples, the local gallery may include a subset of biometric data stored by the identity system. In various examples, the local gallery may be stored by the identity system. In a number of examples, the local gallery may be stored local to the measurement device. In some examples, the identity system may provide the authentication in response to receiving a digital representation of a biometric for the person from the application.

In various implementations, a system for medical screening entry may include an application operable to execute on a request device, a screening server, an identity system, and a measurement device. The application may receive a request for screening from a person. The application may communicate with the identity system to obtain an authentication for the person and add information for the person to a local gallery. The application may communicate with the screening server to perform a screening interaction wherein the application may provide the authentication to the screening server. The measurement device may use the local gallery to obtain an identification for the person. The measurement device may obtain a measurement from the person. The measurement device may register the measurement with a screening record for the person. The measurement device may communicate with an access control station to control access.

In some examples, the measurement device may be integrated into the access control station. In a number of examples, the measurement device may communicate the identification to the screening server to determine the screening record with which to register the measurement. In various examples, the measurement device may communicate the identification to the screening server to determine the measurement to obtain.

In a number of examples, the measurement device may use the local gallery upon obtaining a digital representation of a biometric for the person. In some such examples, the digital representation of the biometric may be at least one of a retinal image, an iris image, or a facial image.

Although the above illustrates and describes a number of embodiments, it is understood that these are examples. In various implementations, various techniques of individual embodiments may be combined without departing from the scope of the present disclosure.

As described above and illustrated in the accompanying figures, the present disclosure relates to medical screening systems. One or more request devices, screening servers, and measurement and/or other testing devices communicate to receive one or more requests for screening, perform the screening, obtain measurements and/or test results for the screening, and allow and/or deny access based on the screening. In some examples, the testing devices may be home test kits. In various examples, the measurements and/or test results may be verified and/or verified as corresponding to a particular identity. In a number of examples, different confidence levels may be determined using different measurements and/or test results and different access may be allowed and/or denied based on the different determined confidence levels.

The present disclosure recognizes that biometric and/or other personal data is owned by the person from whom such biometric and/or other personal data is derived. This data can be used to the benefit of those people. For example, biometric data may be used to conveniently and reliably identify and/or authenticate the identity of people, access securely stored financial and/or other information associated with the biometric data, and so on. This may allow people to avoid repeatedly providing physical identification and/or other information.

The present disclosure further recognizes that the entities who collect, analyze, store, and/or otherwise use such biometric and/or other personal data should comply with well-established privacy policies and/or privacy practices. Particularly, such entities should implement and consistently use privacy policies and practices that are generally recognized as meeting or exceeding industry or governmental requirements for maintaining security and privately maintaining biometric and/or other personal data, including the use of encryption and security methods that meets or exceeds industry or government standards. For example, biometric and/or other personal data should be collected for legitimate and reasonable uses and not shared or sold outside of those legitimate uses. Further, such collection should occur only after receiving the informed consent. Additionally, such entities should take any needed steps for safeguarding and securing access to such biometric and/or other personal data and ensuring that others with access to the biometric and/or other personal data adhere to the same privacy policies and practices. Further, such entities should certify their adherence to widely accepted privacy policies and practices by subjecting themselves to appropriate third party evaluation.

Additionally, the present disclosure recognizes that people may block the use of, storage of, and/or access to biometric and/or other personal data. Entities who typically collect, analyze, store, and/or otherwise use such biometric and/or other personal data should implement and consistently prevent any collection, analysis, storage, and/or other use of any biometric and/or other personal data blocked by the person from whom such biometric and/or other personal data is derived.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system for medical screening entry, comprising:
an application operable to execute on a request device;
a screening server; and
a measurement device; wherein:
the application receives a request for screening from a person;
the application communicates with the screening server to perform a screening interaction;
the application receives a measurement identifier from the screening server;
the application provides the measurement identifier to the measurement device;
the measurement device obtains a measurement from the person;
the measurement device registers the measurement with a screening record for the person;
the application receives a screening access identifier from the screening server; and
the application provides the screening access identifier to an access control station.

2. The system of claim 1, wherein the measurement identifier comprises a Quick Read code.

3. The system of claim 1, wherein the screening access identifier comprises a Quick Read code.

4. A system for medical screening entry, comprising:
an application operable to execute on a request device;
a screening server;
an identity system; and
a measurement device; wherein:
the application receives a request for screening from a person;
the application communicates with the identity system to obtain an authentication for the person;
the application communicates with the screening server to perform a screening interaction wherein the application provides the authentication to the screening server;
the application receives a measurement identifier from the screening server;
the application provides the measurement identifier to the measurement device;
the measurement device obtains a measurement from the person;
the measurement device registers the measurement with a screening record for the person;
the application receives a screening access identifier from the screening server; and
the application provides the screening access identifier to an access control station.

5. The system of claim 4, wherein personal health information is omitted from the measurement identifier.

6. The system of claim 4, wherein personal identification information is omitted from the measurement identifier.

7. The system of claim 4, wherein the measurement device obtains the measurement using an integrated sensor.

8. The system of claim 4, wherein the measurement device obtains the measurement using a connected sensor.

9. The system of claim 4, wherein the measurement device obtains the measurement from a manual entry obtained from an operator via an interface.

10. A system for medical screening entry, comprising:
an application operable to execute on a request device;
a screening server;
an identity system; and
a measurement device; wherein:
the application receives a request for screening from a person;
the application communicates with the identity system to obtain an authentication for the person and add information for the person to a local gallery;
the application communicates with the screening server to perform a screening interaction wherein the application provides the authentication to the screening server;
the measurement device uses the local gallery to obtain an identification for the person;
the measurement device obtains a measurement from the person;
the measurement device registers the measurement with a screening record for the person;
the application receives a screening access identifier from the screening server; and
the application provides the screening access identifier to an access control station.

11. The system of claim 10, wherein the local gallery includes a subset of biometric data stored by the identity system.

12. The system of claim 10, wherein the local gallery is stored by the identity system.

13. The system of claim 10, wherein the local gallery is stored local to the measurement device.

14. The system of claim 10, wherein the identity system provides the authentication in response to receiving a digital representation of a biometric for the person from the application.

15. A system for medical screening entry, comprising:
an application operable to execute on a request device;
a screening server;
an identity system; and
a measurement device; wherein:
the application receives a request for screening from a person;
the application communicates with the identity system to obtain an authentication for the person and add information for the person to a local gallery;
the application communicates with the screening server to perform a screening interaction wherein the application provides the authentication to the screening server;
the measurement device uses the local gallery to obtain an identification for the person;
the measurement device obtains a measurement from the person;
the measurement device registers the measurement with a screening record for the person; and
the measurement device communicates with an access control station to control access.

16. The system of claim 15, wherein the measurement device is integrated into the access control station.

17. The system of claim 15, wherein the measurement device communicates the identification to the screening server to determine the screening record with which to register the measurement.

18. The system of claim 15, wherein the measurement device communicates the identification to the screening server to determine the measurement to obtain.

19. The system of claim 15, wherein the measurement device uses the local gallery upon obtaining a digital representation of a biometric for the person.

20. The system of claim 19, wherein the digital representation of the biometric comprises at least one of a retinal image, an iris image, or a facial image.
